(19) United States
(12) Patent Application Publication
(10) Pub. No.: US 2017/0215696 A1
(43) Pub. Date: Aug. 3, 2017

(54) ENDOSCOPY SYSTEMS AND RELATED METHODS

(71) Applicant: Boston Scientific Scimed, Inc., Maple Grove, MN (US)

(72) Inventors: Timothy P. Harrah, Cambridge, MA (US); Christopher L. OSKIN, Grafton, MA (US); Derrick LENZ, Pompton Plains, NJ (US); Arpita BANERJEE, Bangalore (IN); Abhijit TAKALE, Pune (IN); Pavan MISRA, Bangalore (IN); Sandesh GAVADE, Bangalore (IN)

(73) Assignee: Boston Scientific Scimed, Inc., Maple Grove, MN (US)

(21) Appl. No.: 15/415,676

(22) Filed: Jan. 25, 2017

Related U.S. Application Data
(60) Provisional application No. 62/288,899, filed on Jan. 29, 2016.

(51) Int. Cl. A61B 1/005; A61B 1/018; A61B 18/24; A61B 1/00

(52) U.S. Cl. CPC A61B 1/0052; A61B 1/0057; A61B 1/0055; A61B 1/0016; A61B 1/00128; A61B 1/018; A61B 18/24; A61B 2017/0034

(57) ABSTRACT

According to aspects of the present disclosure, an endoscope may include a handle including a control mechanism. The endoscope may also include an adapter spaced from and coupled to the handle by a flexible connector. The endoscope may further include a flexible shaft coupled to the adapter. The control mechanism may be operatively coupled to the shaft to cause movement of the shaft.

### CROSS-REFERENCE TO RELATED APPLICATIONS

This application claims the benefit of priority of U.S. Provisional Application No. 62/288,899, filed Jan. 29, 2016, the entirety of which is incorporated by reference into this application.

### TECHNICAL FIELD

Various aspects of the present disclosure relate generally to endoscopy systems and related methods. More specifically, the present disclosure relates to control features of endoscopy systems and related methods.

### BACKGROUND

The field of endoscopy covers systems and methods a user may employ to examine and/or treat a subject with, e.g., the assistance of an endoscope or other suitable introduction sheaths or devices. An endoscope (or other suitable introduction device) may provide for viewing of, for example, the interior of a hollow organ or cavity in the subject's body. Ureteroscopy and pyeloscopy are subsets of endoscopy. Ureteroscopy may include procedures in which an endoscope (such as, e.g., a ureteroscope) may be passed through the subject's urethra and bladder, and directly into the subject's ureter. The endoscope may be further inserted into the subject's kidney for pyeloscopic procedures. These procedures may be useful in the diagnosis and the treatment of disorders of the subject's urinary tract, such as the presence of kidney stones that may block urinary tract ducts. Many approaches are available for treating such stones, including, for example, laser treatment and subsequent removal of the stones from the body. Enhancing control of the endoscope during performance of such procedures may lead to improved outcomes.

### SUMMARY

Aspects of the present disclosure relate to, among other things, a endoscopy systems and related methods. Each of the aspects disclosed herein may include one or more of the features described in connection with any of the other disclosed aspects.

In one aspect of the present disclosure, an endoscope may include a handle including a control mechanism. The endoscope may also include an adapter spaced from and coupled to the handle by a flexible connector. The endoscope may further include a flexible shaft coupled to the adapter. The control mechanism may be operatively coupled to the shaft to cause movement of the shaft.

Aspects of the endoscope may include one or more of the features below. The control mechanism may be operatively coupled to the shaft by a steering cable extending from the handle into the shaft via the adapter and the flexible connector. The flexible connector may include an elongate tubular sheath having a lumen, and the steering cable may extend through the lumen. The control mechanism may be operatively coupled to the shaft by an actuator in the handle. The actuator may include an electric motor configured to push and/or pull the steering cable. The adapter may include a rigid body having a proximal end coupled to the connector and a distal end coupled to the shaft. The adapter may include a central lumen in communication with the shaft and the connector. The adapter may include a fluid port for receiving fluid. The adapter may include an instrument port for receiving an instrument. The instrument may include an optical fiber. The adapter may include a control mechanism operatively coupled to the shaft, and the control mechanism of the adapter may be operatively coupled to the shaft by an actuator configured to rotate the shaft about a central longitudinal axis of the shaft. The actuator may include an electric motor configured to rotate the shaft relative to a distal end of the adapter.

In another aspect of the present disclosure, an endoscope may include a handle, a flexible shaft, and an adapter between the handle and the shaft. The adapter may be coupled to the handle by a flexible connector. The adapter may include a rigid body, a first port on the body for receiving a fluid, a second port on the body for receiving an instrument, and an actuator coupled to the body. The actuator may be configured to engage the instrument and move the instrument relative to the body when the actuator is activated.

Aspects of the endoscope may include one or more of the features below. The actuator may further include a control mechanism for controlling activation of the actuator. The control mechanism may include a switch. Depressing a first portion of the switch may activate the actuator to move the instrument in a first direction. Depressing a second portion of the switch may activate the actuator to move the instrument in a second direction. The second direction may be opposite the first direction. The shaft may include a lumen in communication with the first port, and fluid received in the first port may flow into the lumen. The shaft may include a lumen in communication with the second port, and the instrument received in the second port may extend into the lumen. The lumen may terminate at an opening at a distal end of the shaft.

In another aspect of the present disclosure, a method for performing a procedure using an endoscope may include inserting an endoscope into a subject. The endoscope may include a flexible shaft configured for insertion into the subject, and an adapter coupled to the shaft. The adapter may be configured to remain outside of the subject. The endoscope may also include a handle coupled to the adapter by a flexible connector. The handle may be configured to remain outside of the subject. The method may also include moving the shaft via a control mechanism on the handle to position a distal end of the shaft in a target area inside the subject. The method may also include positioning a distal end of an instrument at the target area by inserting the instrument into a port of the adapter, and actuating a control mechanism on the actuator to move the instrument.

Aspects of the method for performing the procedure using the endoscope may include one or more of the features below. Inserting the endoscope into the subject includes sliding the shaft and the adapter in the direction of the subject. The adapter may be mounted above a surface of a table by a support member. The support member may be coupled to the table via a sliding mechanism that provides the ability to translate the support member toward and away from the subject.

It may be understood that both the foregoing general description and the following detailed description are exemplary and explanatory only and are not restrictive of the features claimed.

As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.”

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1 is a perspective view of part of an endoscopy system including an endoscope, in accordance with aspects of the present disclosure.

FIG. 2 is a perspective view of a handle system of the endoscope of FIG. 1, in accordance with aspects of the present disclosure.

FIG. 3 is a perspective view of a portion of the handle system of FIG. 2, in accordance with aspects of the present disclosure.

FIG. 4 is a perspective view of an adapter of the endoscope of FIG. 1, in accordance with aspects of the present disclosure.

FIG. 5 is a cross-sectional view of the adapter of FIG. 4, in accordance with aspects of the present disclosure.

FIG. 6 is another perspective view of part of an endoscopy system including an endoscope and a control unit, in accordance with aspects of the present disclosure.

FIG. 7 is a schematic depiction of an endoscopy system, in accordance with aspects of the present disclosure.

### DETAILED DESCRIPTION

The present disclosure is drawn generally to endoscopy systems and related methods, and more specifically to control features of endoscopy systems and related methods. Reference now will be made in detail to aspects of the present disclosure, examples of which are illustrated in the accompanying drawings. Wherever possible, the same reference numbers will be used throughout the drawings to refer to the same or like parts. The term “distal” refers to a portion farthest away from a user when introducing an instrument into a subject. By contrast, the term “proximal” refers to a portion closest to the user when placing the instrument into the subject. Though the following description refers to “endoscope” or “endoscopy,” the principles/aspects described herein may be used with any suitable introduction sheath or device, even if such sheath or device fails to include one or more features typically associated with “endoscopes.”

### Endoscopy System

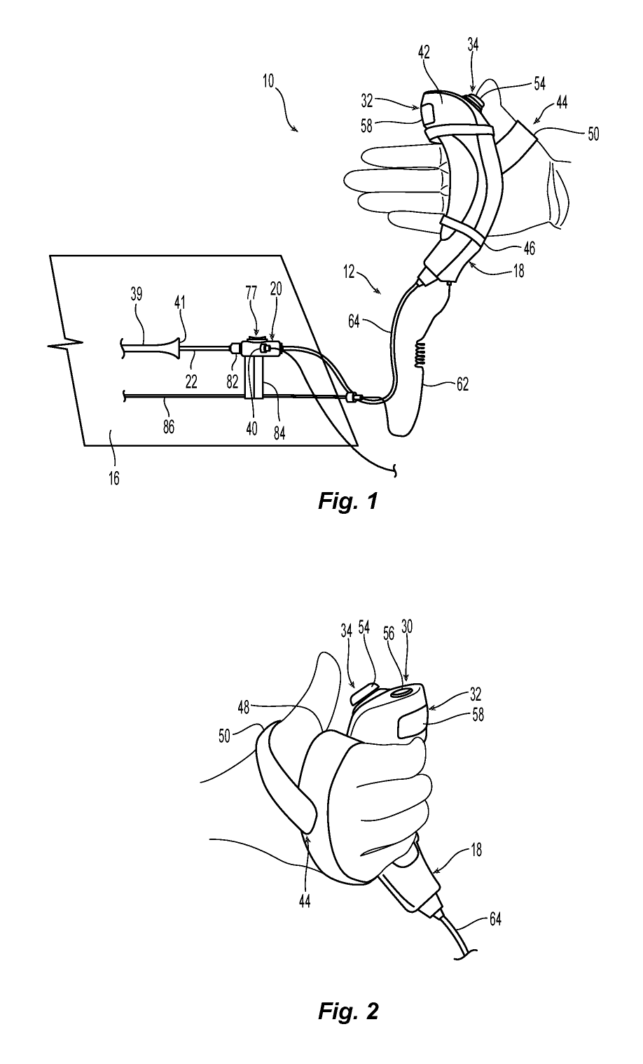

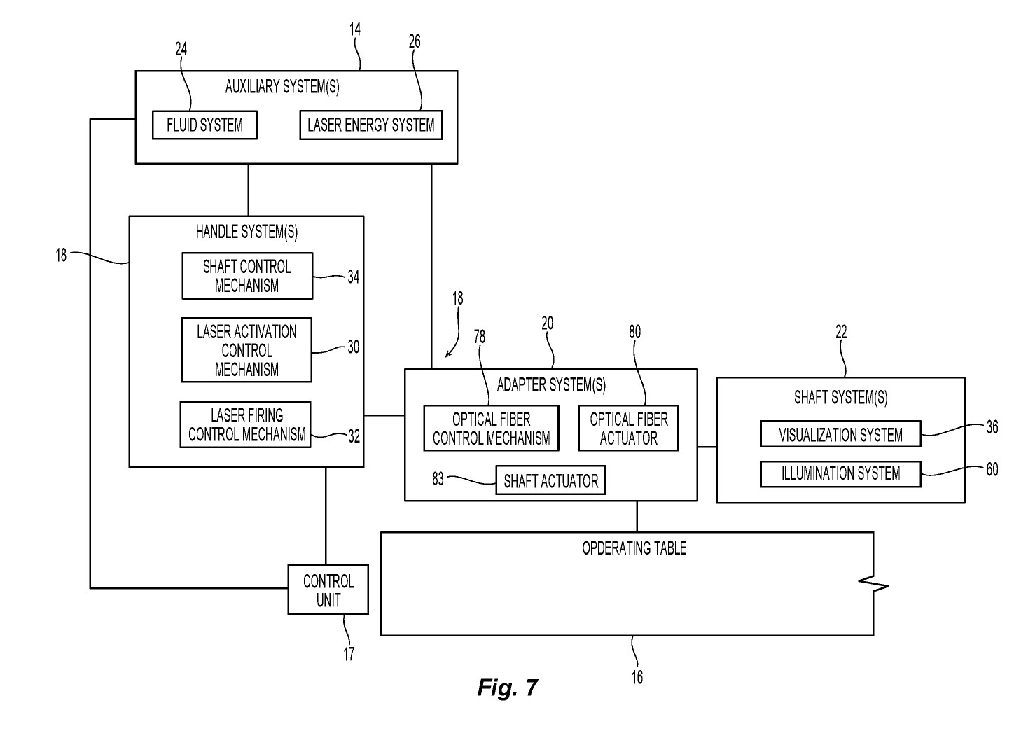

FIGS. 1 and 7 show aspects of an exemplary endoscopy system 10. System 10 may include an endoscope 12, one or more auxiliary systems 14 operatively coupled to endoscope 12, an operating table 16 for supporting a subject, and a control unit 17 operatively coupled to endoscope 12 and/or auxiliary systems 14. Endoscope 12, either alone or with the assistance of control unit 17 and auxiliary systems 14, may be used to examine and/or treat the subject.

Endoscope 12 may include a handle 18, an adapter 20, and a shaft 22. Auxiliary systems 14 may include, for example, a fluid system 24 and/or a laser energy system 26. Fluid system 24 may direct a fluid, such as saline, into endoscope 12. For example, fluid system may direct fluid into shaft 22 via adapter 20. Fluid system 24 also may be configured to provide insufflation gases and/or vacuum suction, as desired for certain applications/procedures. Laser energy system 26 may supply laser energy into endoscope 12. For example, laser energy system 26 may direct laser energy into shaft 22 via adapter 20. Laser energy system 26 may be replaced with any suitable energy delivery system, including but not limited to optical, thermal, or electromagnetic (e.g., radiofrequency energy).

Handle 18 may be gripped by a user's hand. Handle 18 may be used to control one or more components of endoscopy system 10. For example, handle 18 may be used to control the application of laser energy from laser energy system 26. This control may be provided by a laser activation control mechanism 30 and a laser firing control mechanism 32 on handle 18. In one example, laser activation control mechanism 30 may be used to signal laser energy system 26, e.g., to prepare laser energy system 26 for firing laser energy. Laser firing control mechanism 32 may be used to fire the laser energy at a suitable target.

Handle 18 also may be used to control movement or orientation of shaft 22. For example, handle 18 may include a shaft control mechanism 34 for deflecting and/or rotating shaft 22. In use, at least a portion of shaft 22 of endoscope 12 may be inserted into the subject by the user while at least handle 18 remains outside of the subject, allowing the user to manipulate shaft control mechanism 34 to guide the insertion and/or otherwise position shaft 22 in the subject. Additionally or alternatively, shaft control mechanism 34 may move shaft 22 to bring a target area in the subject's body within a field of view of a visualization system 36 within shaft 22 (e.g., within the distal end of shaft 22). Additionally or alternatively, shaft control mechanism 34 may move shaft 22 to facilitate aiming of laser energy from laser energy system 26 that may be emitted from the distal end of shaft 22. Such shaft movements may provide the user with multiple degrees of control over the positioning of shaft 22, which may be useful during examination and/or treatment of the subject.

With reference to FIGS. 1 and 4, adapter 20 of endoscope 12 may be operatively coupled to handle 18 and shaft 22. For example, adapter 20 may form at least part of a connection between handle 18 to shaft 22. Adapter 20 may include a fluid port 38 through which the fluid from fluid system 24 may flow into adapter 20. Adapter 20 also may include a laser energy port 40 through which laser energy may be directed into adapter 20 via, for example, an optical fiber 76 or other laser energy conductor. The fluid and/or laser energy may pass through adapter 20 on its way into shaft 22, and then out of the distal end of shaft 22 and into the subject's body. Alternatively, it is contemplated that port 40 may receive a retrieval device (e.g., a basket or a forceps) instead of a laser energy conductor. Alternatively, one or more additional ports (not shown) that may be similar to port 40 may be provided, with each of the ports being capable of receiving one of a laser energy conductor, retrieval device, or other suitable instrument.

According to one aspect of the present disclosure, endoscopy system 10 may be used to diagnose and/or remove kidney stones in the subject. To do so, shaft 22 of endoscope 12 may be inserted into the subject's urethra, bladder, or ureter. Such insertion may be facilitated by use of an introduction sheath 39 (e.g., a ureteral sheath). It is also contemplated that shaft 22 may be further inserted beyond the ureter into the subject's kidney. Once in place, the laser energy from laser energy system 26 and/or the fluid from fluid system 24 may be used to treat any stones. Treatment may include fragmenting the stones and/or removing them. It should be understood, however, that endoscope 12 may be used to examine and/or treat any internal area of the subject.

### Handle

Handle 18 may include a rigid housing 42 having an exterior surface shaped and/or textured to facilitate gripping by the user in, e.g., a single hand. Housing 42 may be rigid in that it may not bend, buckle, or otherwise deform when gripped or otherwise manipulated by the user. Handle 18 also may include a flexible strap 44 for securing handle 18 to the user's hand. Strap 44 may include one or more handle connection members 46 extending around one or more portions of housing 42. Strap 44 also may include a hand connection member 48 extending from handle connection member 46 and configured to extend around the back of the user's hand and/or knuckles (see FIGS. 1 and 2). Strap 44 may further include a thumb connection member 50 extending from at least one of handle connection member 46 and hand connection member 48, and around the thumb of the user's hand. Thumb connection member 50 may pivot relative to handle connection member 46 and/or hand connection member 48. It is contemplated that strap 44 may be sufficiently flexible so as to elastically deform for facilitating mounting of strap 44 to handle 18 and/or removing strap 44 from handle 18. By securing strap 44 to the user's hand, the user's other hand may not be needed to support handle 18, and thus, may remain free to perform other tasks. Further, the user may release his or her grip of handle 18 without dropping handle 18, allowing the user to use his fingers and/or thumb to perform other tasks while still supporting handle 18 via strap 44. However, it should be understood that strap 44 may be omitted. Handle 18 is shown in FIG. 6 without strap 44.

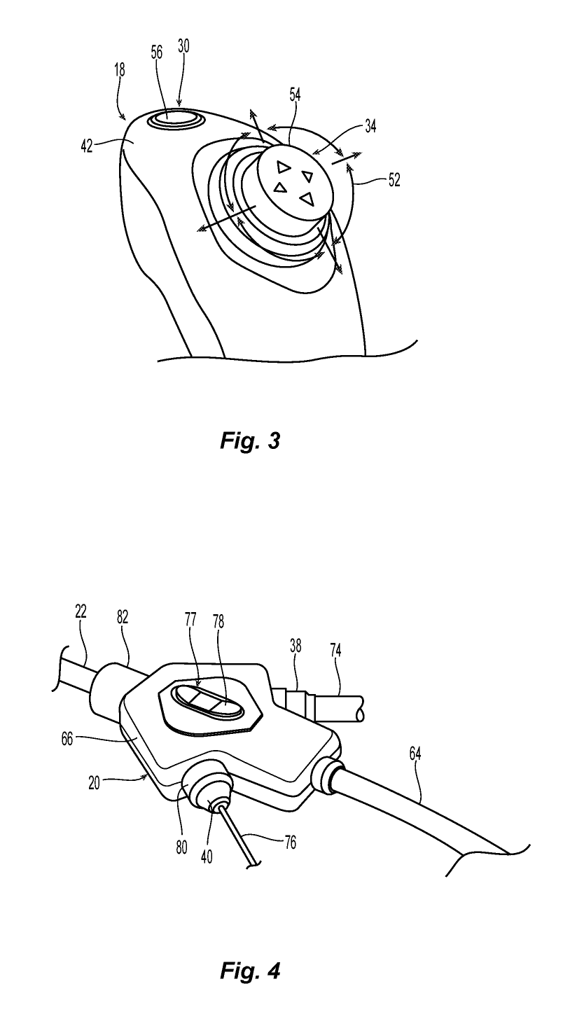

Shaft control mechanism 34 of handle 18 may include a joystick 54, shown up close in FIG. 3, projecting from the external surface of housing 42 at a proximal end thereof. Joystick 54 may be positioned such that, when handle 18 is positioned against the user's palm, joystick 54 may be manipulated by the user's thumb. Joystick 54 may pivot, rotate, or otherwise articulate in or more directions relative to housing 42, with such movement being translated into movement of shaft 22. For example, joystick 54 may be operatively coupled to proximal ends of one or more steering cables (not shown). The steering cables may extend into shaft 22, with distal ends of the steering cables being operatively coupled to the distal end of shaft 22. Movement of joystick 54 may push and/or pull the steering wires to tilt, bend, or otherwise deflect shaft 22. In one example, movement of joystick 54 in up and down directions may result in deflection of shaft 22 along the y-axis, and movement of joystick 54 in left and right directions may result in deflection of shaft 22 along the x-axis. Directional arrows 52 portray exemplary movements of joystick 54 in the up, down, left, and right directions, and arcs between those directions.

The operative coupling between joystick 54 and the steering cables may include one or more mechanical components or devices (not shown), such that forces exerted on joystick 54 by the user may push and/or pull the steering cables. The mechanical components/devices may, for example, be contained within housing 42 of handle 18. Alternatively, the operative coupling between joystick 54 and the steering cables may include one or more electronic components or devices (not shown). The electronic components/devices may include, for example, a joystick monitoring device for monitoring movement of joystick 54, and sending one or more output signals based on the movement. The electronic components/devices also may include one or more actuators, such as electric motors, that may receive the output signals. The actuators may be operatively coupled to the steering cables such that the electric motors may push and/or pull the steering cables based on the output signals. It is also contemplated that joystick 54 may be twisted in clockwise and counterclockwise directions, triggering the joystick monitoring device to send one or more output signals to one or more actuators that may rotate shaft 22 about its central longitudinal axis. This rotation will be described in greater detail below. The above-described electronic components/devices may be contained within or otherwise attached to housing 42 and/or adapter 20. For example, the joystick monitoring device and/or the push/pull actuators may be contained within or otherwise attached to housing 42, and the rotation actuators may be contained within or otherwise attached to adapter 20.

Laser activation control mechanism 30 may include a switch or button 56 at the proximal end of housing 42, such that button 56 may be depressed by the user's thumb when handle 18 is positioned against the palm of the user's hand. When button 56 is depressed, laser activation control mechanism 30 may send an output signal to laser energy system 26, instructing laser energy system 26 to prepare to fire laser energy. As an added safety measure, button 56 may be have to be depressed for a predetermined amount of time (e.g., 2 seconds) before laser energy system 26 is armed. Once laser energy system 26 is armed, it may be prepared to fire a continuous stream of the laser energy. Alternatively, laser energy system 26 may be prepared to fire one or more bursts of the laser energy.

Laser firing control mechanism 32 of handle 18 may include a switch or button 58 at the proximal end of housing 42 of handle 18. For example, button 58 may be positioned on a side of housing 42 such that button 58 may be depressed by the user's pointer finger when handle 18 is against the palm of the user's hand. Once laser energy system 26 is armed, depressing button 58 may cause laser energy system 26 to fire laser energy. Buttons 56 and 58 may have to be depressed simultaneously to fire the laser energy. Alternatively, the user may depress button 56 to arm laser energy system 26, and then may depress button 58 to fire the laser energy, whether or not button 56 remains depressed. Having the user depress two buttons 56 and 58 in order to fire the laser energy may reduce the risk of the user inadvertently firing the laser energy at unintended targets in the subject's body.

It is contemplated that the laser energy may be fired in different modes. For example, in one mode of operation, the laser energy may continue to fire in bursts of predetermined duration or in a continuous stream as long as button 58 remains depressed. This may allow the user to deliver a large amount of laser energy in a short period of time. Additionally or alternatively, in another mode, the laser energy may fire in a single burst of predetermined duration regardless of whether or not button 58 remains depressed. This may help safeguard against accidentally discharging the laser energy at an unintended target due to unexpected movement of endoscope 12 or the subject that may throw off the user's aim. Additionally or alternatively, fully depressing button 58 may fire a continuous stream of the laser energy, while depressing button 58 halfway may fire one or more bursts of predetermined duration. Additionally or alternatively, depressing button 58 while depressing button 56 may fire a continuous stream of the laser energy, while depressing button 58 alone (after arming laser energy system 26 with button 56) may fire one or more bursts of predetermined duration. Additionally or alternatively, in another mode of operation, laser energy system 26 may not fire a subsequent burst or stream of laser energy for a predetermined period of time after an initial firing. This may ensure that there is sufficient time for heat to be dissipated from the target area, thus protecting tissue from heat damage, before any additional firing of the laser energy.

### Control Unit

Shaft control mechanism 34, laser activation control mechanism 30, laser firing control mechanism 32, and/or any of the above-described electronic components/devices may be operatively coupled to control unit 17 (FIGS. 6 and 7), such that signals sent to and/or received from these components may be generated by, processed by, and/or sent through control unit 17. Control unit 17 also may be operatively coupled to auxiliary systems 14, providing a communication link between, for example, endoscope 12 and laser energy system 26. The communication link may be in the form of wired and/or wireless communications. Control unit 17 may be any suitable computing device. It is also contemplated that control unit 17 may include, or may be operatively coupled to, a power supply, such that control unit 17 may deliver power to endoscope 12 to operate the powered components/devices therein.

Control unit 17 may be separate from endoscope 12, auxiliary systems 14, and operating table 16. In one example, handle 18 of endoscope 12 may be connected to control unit 17 by a flexible connector 62. Connector 62 may be flexible in that it may bend, elongate, or otherwise deform during use. Alternatively, control unit 17 may be directly incorporated into one or more of endoscope 12, auxiliary systems 14, and operating table 16. For example, at least a portion of control unit 17 (e.g., a circuit board operatively coupled to shaft control mechanism 34, laser activation control mechanism 30, laser firing control mechanism 32, and/or any of the above-described electronic components/devices in endoscope 12) may be incorporated into handle 18 of endoscope 12. Another portion of control unit 17 (e.g., a computing device and communications link) may be external to handle 18.

### Adapter and Shaft

A flexible connector 64 may link handle 18 to adapter 20. For example, connector 64 may connect a distal end of housing 42 to a proximal end of adapter 20. Connector 64 may include a tubular outer sheath defining one or more lumens (not shown) containing components (e.g., mechanical and/or electrical) such as, for example, the steering cables, one or more cables/wires operatively linking the joystick monitoring device and the actuators, and/or one or more cables/wires for transmitting power from handle 18 to powered components of adapter 20 and shaft 22. Connector 64 may be flexible in that it may bend or otherwise deform during use.

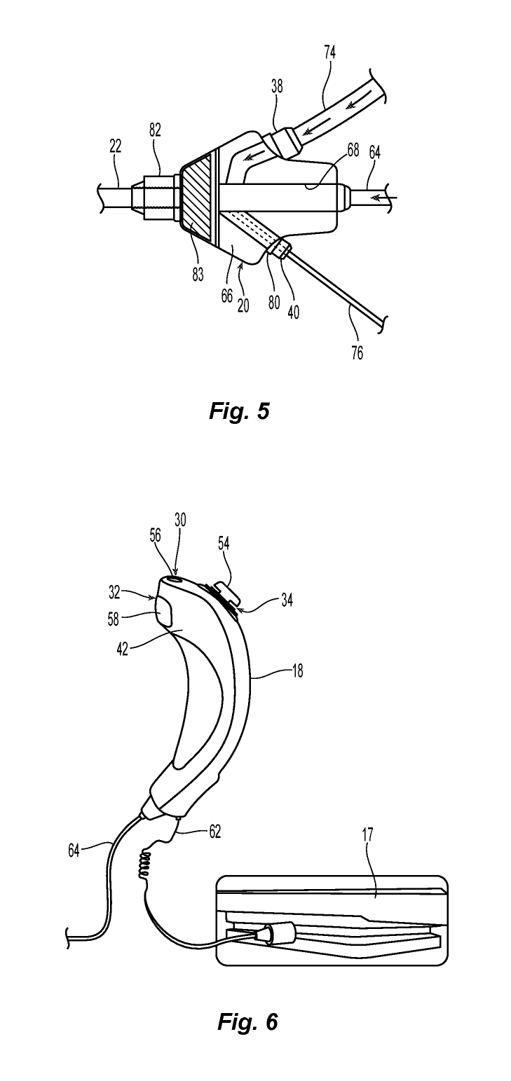

As shown in greater detail in FIGS. 4 and 5, adapter 20 may include a rigid body 66 having a proximal end configured to receive the distal end of connector 64, and a distal end configured to receive the proximal end of shaft 22. Body 66 may be rigid in that it may not bend, buckle, or otherwise deform during use. One or more central lumen(s) 68 may extend between the proximal and distal ends of body 66. The steering cables and/or wires in connector 64 may extend into and through central lumen(s) 68 on their way to shaft 22.

Body 66 may also include fluid port 38 on one side and laser energy port 40 on another side. Though only a single fluid port 38 and a single laser energy port 40 is depicted, body 66 may include any suitable number of ports. Fluid port 38 may be in fluid communication with fluid system 24. For example, a fluid tube 74 may extend from fluid system 24 to fluid port 38. Fluid port 38 may permit the fluid to enter body 66 and flow through body 66 on its way to shaft 22. According to one aspect, fluid port 38 may permit the fluid to enter central lumen(s) 68, from which the fluid may then be directed into shaft 22 as shown in FIG. 5. Laser energy port 40 may receive laser energy from laser energy system 26. For example, an optical fiber 76, or any other suitable laser energy transmitting component, may extend from laser energy system 26 into laser energy port 40. Upon entering laser energy port 40, optical fiber 76 may enter central lumen(s) 68. From central lumen(s) 68, optical fiber 76 may enter shaft 22. The laser energy from laser energy system 26 may be transmitted through optical fiber 76, and emitted from the distal end of optical fiber 76. While two ports 38 and 40 can be seen in FIGS. 4 and 5, it is contemplated that at least one of the two ports 38 and 40 may be omitted. Alternatively, additional ports may be provided on body 66 to receive additional instruments.

Body 66 of adapter 20 may also include an optical fiber control mechanism 77. Optical fiber control mechanism may include a switch 78, such as a rocker switch, that may be operatively coupled to an actuator 80. Actuator 80 may include, for example, an electric motor housed within or otherwise attached to body 66. Actuator 80 may engage the external surface of optical fiber 76. When a first side of switch 78 is depressed by the user, actuator 80 may be activated to drive optical fiber 76 in the distal direction. When a second side of switch 78 is depressed, actuator 80 may be activated to drive optical fiber 76 in the proximal direction. It is contemplated that switch 78, when depressed, may continue to move optical fiber 76 until switch 78 is allowed to return to a rest position. Alternatively, optical fiber 76 may move a predetermined distance when switch 78 is depressed. Further incremental movements of optical fiber 76 may be carried out by keeping switch 78 depressed, or by allowing switch 78 to return to its rest position, and then depressing switch 78 again. Alternatively, depressing switch 78 halfway may cause optical fiber 76 to move in predetermined increments of distance, and depressing switch 78 fully may cause optical fiber 76 to move continuously as long as switch 78 remains fully depressed. Such features may assist with fine adjustment of the position of the distal end of optical fiber 76.

When switch 78 is at rest, actuator 80 may lock optical fiber 76 in place. Alternatively, when switch 78 is at rest, actuator 80 may be disengaged from optical fiber 76, allowing the user to move optical fiber 76 proximally and distally manually. In yet another alternative, switch 78 may be omitted. Actuator 80 may instead include a knob or wheel (not shown) that engages the external surface of optical fiber 76 via any suitable mechanism known in the art. The user may manually rotate the knob/wheel to drive optical fiber 76 in the proximal and distal directions. Control over the proximal/distal movement of optical fiber 76 may help the user aim the laser energy emitted from the distal end of optical fiber 76 with greater precision than would otherwise be the case.

Shaft 22 may extend distally from the distal end of body 66. Shaft 22 may include one or more lumens (not shown) in communication with central lumen(s) 68 of adapter 20. For example, shaft 22 may include a fluid lumen extending between its proximal and distal ends, for receiving the fluid introduced into endoscope 12 via fluid port 38. The fluid may be emitted out of an opening at the distal end of shaft 22. Shaft 22 may include a laser energy lumen extending between its proximal and distal ends, for receiving optical fiber 76. Optical fiber 76 may be inserted through the laser energy lumen until the distal (laser-emitting) end of optical fiber 76 extends to or distally out of an opening at the distal end of shaft 22. At least a portion of shaft 22 may be flexible, in that the portion may bend, twist, elongate, compress, or otherwise deform during use. For example, at least the distal portion of shaft 22 may be flexible.

According to one aspect, shaft 22 may be rotatably coupled to the distal end of body 66 by a rotational coupling 82, such that shaft 22 may rotate relative to the distal end of body 66 about the central longitudinal axis of shaft 22. An actuator 83 (FIG. 5), such as an electric motor, may be contained within or otherwise attached to body 66, to drive rotation of shaft 22. Actuator 83 may be activated by joystick 54. Twisting (e.g., rotating) joystick 54 in one of a clockwise and counterclockwise direction may trigger actuator 83 to rotate shaft 22 in the same direction. Twisting joystick 54 in the other direction may trigger actuator 83 to rotate shaft 22 in that direction. Alternatively, actuator 83 may be activated by a switch (not shown), similar to switch 78, provided on body 66. As another alternative, actuator 83 may include a knob or wheel (not shown) that engages shaft 22, and may be manually actuated to rotate shaft 22. Rotating shaft 22 may provide the user with an additional degree of control over the positioning of the distal end of shaft 22.

Shaft 22 and adapter 20 may be supported on operating table 16 by a support member 84 (FIG. 1). Support member 84 may have an upper end engaging a lower surface of adapter 20, and a lower end engaging an upper surface of operating table 16. As such, adapter 20 may act as a proximal base for shaft 22, in place of handle 18, allowing shaft 22 to be shorter than would otherwise be the case. The shorter length of shaft 22 may make it easier to control by, for example, reducing the amount of force needed to deflect shaft 22. Additionally or alternatively, the user may be able to move handle 18, or even put handle 18 down, without inadvertently moving shaft 22, thus reducing user fatigue that could result from the user holding handle 18 in a fixed position for a long period of time while performing a procedure. Further, by supporting shaft 22 and adapter 20, support member 84 may allow the user to keep one of his or her hands free and ready to perform other tasks. Furthermore, support member 84 may provide adapter 20 and shaft 22 with stability so that precision movements of the distal end of shaft 22 may be performed.

It is contemplated that support member 84 may be adjustable to accommodate different subjects. For example, support member 84 may be adjustable in height, for example by using telescoping parts, to selectively position adapter 20 and shaft 22 at different distances from the top surface of operating table 16. Additionally or alternatively, support member 84 may be one of a plurality of support members, each having a different height, where one support member may be swapped out for another.

According to one aspect, the lower end of support member 84 may be movably coupled to operating table 16. For example, the lower end may be slidably coupled to a rail 86 or other protrusion on operating table 16. Rail 86 may also provide a lumen through which connector 62 may pass on its way from handle 18 to control unit 17. Alternatively, the lower end may be slidably received within a slot (not shown) or other depression on operating table 16. Support member 84 and adapter 20 may slide proximally to withdraw shaft 22 from the subject, and distally to insert shaft 22 into the subject. A drive mechanism (not shown) may be provided in operating table 16 to slide support member 84. The drive mechanism may be activated by a switch (not shown), similar to switch 78, provided on operating table 16, adapter 20, or handle 18.

It is also contemplated that endoscope 12 may be used without support member 84. In such a scenario, adapter 20 may rest on the top surface of operating table 16 while shaft 22 is in the subject. The user may pick up adapter 20 with his or her free hand to insert shaft 22 and withdraw shaft 22, and to manipulate optical fiber control mechanism 77 and/or any other controls on body 66 of adapter 20.

As described above, shaft 22 may include one or more lumens (not shown) extending between its proximal and distal ends, with the lumens being in communication with central lumen(s) 68 of adapter 20. The lumens of shaft 22 may include a fluid lumen for receiving the fluid from adapter 20, and for emitting the fluid out of an opening (not shown) at the distal end of shaft 22. The lumens also may include a laser energy lumen for receiving optical fiber 76. The distal end of optical fiber 76 may be extended distally out of an opening (not shown) at the distal end of shaft 22, such that laser energy emitted from the distal end of optical fiber 76 can be directed at a target. The lumens of shaft 22 may also include one or more steering cable lumens for controlling deflection of shaft 22. A distal end of shaft 22 may include suitable radiopaque markings/features.

The lumens also may include a visualization lumen for receiving a visualization device (not shown), such as a camera assembly, allowing the user to view a target area and control movement of shaft 22. The camera assembly may include a digital camera coupled to the distal end of shaft 22, with a field of view covering an area distal to the distal end of shaft 22. One or more cables/wires (not shown) may extend from the digital camera to handle 18 via shaft 22, adapter 20, and connector 64, and from handle 18 to control unit 17 via connector 62, for supplying the digital camera with power, sending control signals to the digital camera, and receiving image data from the camera. Alternatively, the visualization device may include a fiber optic assembly extending proximally from the distal end of shaft 22 for delivering light (forming an image) to handle 18 and/or control unit 17. Control unit 17 may send image data to an auxiliary display (not shown) for viewing by the user.

The lumens may also include one or more illumination lumens. The illumination lumens may receive illumination system 60, which may include light-emitting diode assemblies, light-transmitting fiber optic assemblies, and/or any other suitable devices for emitting light distally from the distal end of shaft 22 to illuminate a target area. The illumination devices may be in communication with control unit 17, which may control one or more operational parameters of the illumination devices.

### Methods of Use

In use, with the subject resting on the top surface of operating table 16, the user may insert the introduction sheath 39 into the subject's urinary tract. Introduction sheath 39 may have a tapered distal end (not shown) for dilating the ureter, a flared proximal end with an opening 41 for facilitating insertion of shaft 22 into introduction sheath 39, and a lumen extending between the distal and proximal ends for receiving shaft 22. Introduction sheath 39 may extend through the subject's urethra and bladder, such that the distal end of introduction sheath 39 may be positioned in the subject's ureter.

The user may align the distal end of shaft 22 with opening 41. Once aligned, the user may insert shaft 22 into opening 41. The insertion step may include the user using one hand to guide shaft 22 into introduction sheath 39. Additionally or alternatively, the insertion step may include the user sliding adapter 20 and shaft 22 distally along operating table 16, via the slidable coupling provided by support member 84. The user may continue to slide shaft 22 distally through introduction sheath 39 until the distal end of shaft 22 is in the subject's urethra, bladder, or ureter. Additionally, the user may continue to slide shaft 22 so its distal end moves from the ureter into the subject's kidney.

During insertion of shaft 22 and/or positioning of the distal end of shaft 22 at a target area, the user may use handle 18 to manipulate its controls to facilitate insertion. For example, the user may manipulate joystick 54 to deflect and/or rotate shaft 22 so that the distal end of shaft 22 can be configured (and reconfigured) for navigation through the subject's anatomy. Additionally or alternatively, the user may view the area distal to the distal end of shaft 22, using the visualization system 36 and/or illumination system 60, to help guide shaft 22 and locate the target area. Because handle 18 may be secured to one of the user's hands by strap 44, the user may be able to release his or her thumb and/or fingers from handle 18, without dropping handle 18. As such, both of the user's hands may be capable of performing other tasks including, for example, handling or otherwise manipulating adapter 20, shaft 22, operating table 16, auxiliary systems 14, introduction sheath 39, and/or the subject.

With the distal end of shaft 22 at the target area, and with the target area in view of the visualization device, the user may perform a procedure. For example, the user may locate a kidney stone. Using joystick 54, the user may deflect and/or rotate the distal end of shaft 22 to aim optical fiber 76 of laser energy system 26 at the kidney stone. Additionally or alternatively, the user may move shaft 22 proximally or distally by moving adapter 20 proximally or distally.

Optical fiber 76 may be inserted into shaft 22 via laser energy port 40 of adapter 20. With optical fiber 76 loaded in shaft 22, the user may manipulate switch 78 to actuate actuator 80. Actuator 80 may move optical fiber 76 towards or away from the kidney stone for aiming purposes, in preparation for firing laser energy from the distal end of optical fiber 76 at the kidney stone. Switch 78 and adapter 20 may provide the user with a mechanism for making gross and fine adjustments to the position of the distal end of optical fiber 76 that may not be easily performed just by translating, rotating, and/or deflecting the distal end of shaft 22.

The user may press button 56 to ready laser energy system to fire laser energy. The user may then press button 58 to fire the laser energy. The laser energy may be emitted toward the kidney stone from the distal end of optical fiber 76, and may fragment the kidney stone. If fragmented into small enough pieces, the fragments of the kidney stone may be flushed out of the target area with the fluid from fluid system 24. That is, the fluid from fluid system 24 may be directed into fluid port 38 of adapter 20 of endoscope 12, from adapter 20 into shaft 22 of endoscope 12, out of the distal end of shaft 22, and into the target area. Alternatively, the fluid from fluid system 24 may be directed into fluid port 38 of adapter 20 of endoscope 12, from adapter 20 into a separate lumen (not shown) external to shaft 22, out of the distal end of the lumen, and into the target area. Alternatively, the fluid may exit from body 66 around shaft 22, and may flow to the target area through introduction sheath 39. The fluid may flush out the target area, and then exit the target area via the ureter (e.g., through the ureter and around introduction sheath 39, through the ureter after removal of introduction sheath 39 and shaft 22), bladder (e.g., during micturition), and urethra, taking the fragments with it. The fluid may also flush out other fluids or solid materials from the target area, keeping clear the user's view of the target area. It is also contemplated that the fluid may generate pressure against the tissue surrounding the target area, thus helping to expand the size of the passage in the ureter and/or the size of one or more cavities in the kidney. This expansion may create space to move shaft 22 and optical fiber 76, thus making it easier to locate and fragment the kidney stone. The expansion may also facilitate flushing of the target area by providing more room for fluid flow.

In some instances, one of more of the fragments may be so large that further fragmentation may be desired. In such an instance, the user may move shaft 22 proximally or distally, rotate shaft 22 clockwise or counterclockwise, deflect shaft 22, and/or move optical fiber 76 proximally or distally, using joystick 54 and/or switch 78, to direct the distal end of optical fiber 76 toward the fragment. The user may then use buttons 56 and 58 to fire laser energy at the fragment and break it apart into even smaller fragments. This process may be repeated until the fragments are small enough to be flushed out of the target area by the fluid. Additionally or alternatively, a separate medical instrument having a grasping mechanism may be inserted into the target area to grasp and remove the fragments. Additionally or alternatively, the stone fragments may be suctioned out of the subject via a vacuum system (not shown).

While a laser lithotripsy procedure taking place in the subject's urinary tract has been described, it should be understood that a different procedure may be performed in a similar or different target area of the subject's body. For example, the target area may include any of the other organs of the subject, and the procedure may include, for example, using energy (e.g., thermal) to cut, ablate, and/or coagulate tissue in the target area.

It will be apparent to those skilled in the art that various modifications and variations can be made in the disclosed systems and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. An endoscope, comprising:
a handle including a control mechanism;
an adapter spaced from and coupled to the handle by a flexible connector; and
a flexible shaft coupled to the adapter, wherein the control mechanism is operatively coupled to the shaft to cause movement of the shaft.

2. The endoscope of claim 1, wherein the control mechanism is operatively coupled to the shaft by a steering cable extending from the handle into the shaft via the adapter and the flexible connector.

3. The endoscope of claim 2, wherein the flexible connector includes an elongate tubular sheath having a lumen, and the steering cable extends through the lumen.

4. The endoscope of claim 2, wherein the control mechanism is operatively coupled to the shaft by an actuator in the handle.

5. The endoscope of claim 4, wherein the actuator includes an electric motor configured to push and/or pull the steering cable.

6. The endoscope of claim 1, wherein the adapter includes a rigid body having a proximal end coupled to the connector and a distal end coupled to the shaft.

7. The endoscope of claim 1, wherein the adapter includes a central lumen in communication with the shaft and the connector.

8. The endoscope of claim 1, wherein the adapter includes a fluid port for receiving fluid.

9. The endoscope of claim 1, wherein the adapter includes an instrument port for receiving an instrument.

10. The endoscope of claim 9, wherein the instrument includes an optical fiber.

11. The endoscope of claim 1, wherein the adapter includes a control mechanism operatively coupled to the shaft, and the control mechanism of the adapter is operatively coupled to the shaft by an actuator configured to rotate the shaft about a central longitudinal axis of the shaft.

12. The endoscope of claim 11, wherein the actuator includes an electric motor configured to rotate the shaft relative to a distal end of the adapter.

13. An endoscope, comprising:
a handle;
a flexible shaft; and
an adapter between the handle and the shaft, wherein the adapter is coupled to the handle by a flexible connector, and the adapter includes:
a rigid body,
a first port on the body for receiving a fluid,
a second port on the body for receiving an instrument, and
an actuator coupled to the body, wherein the actuator is configured to engage the instrument and move the instrument relative to the body when the actuator is activated.

14. The endoscope of claim 13, wherein the actuator further includes a control mechanism for controlling activation of the actuator.

15. The endoscope of claim 14, wherein the control mechanism includes a switch, depressing a first portion of the switch activates the actuator to move the instrument in a first direction, and depressing a second portion of the switch activates the actuator to move the instrument in a second direction, the second direction being opposite the first direction.

16. The endoscope of claim 13, wherein the shaft includes a lumen in communication with the first port, and fluid received in the first port flows into the lumen.

17. The endoscope of claim 13, wherein the shaft includes a lumen in communication with the second port, and the instrument received in the second port extends into the lumen.

18. The endoscope of claim 17, wherein the lumen terminates at an opening at a distal end of the shaft.

19. A method for performing a procedure using an endoscope, the method comprising:
inserting an endoscope into a subject, wherein the endoscope includes:
a flexible shaft configured for insertion into the subject,
an adapter coupled to the shaft, the adapter configured to remain outside of the subject, and
a handle coupled to the adapter by a flexible connector, the handle configured to remain outside of the subject;
moving the shaft via a control mechanism on the handle to position a distal end of the shaft in a target area inside the subject; and
positioning a distal end of an instrument at the target area by inserting the instrument into a port of the adapter, and actuating a control mechanism on the actuator to move the instrument.

20. The method of claim 19, wherein inserting the endoscope into the subject includes sliding the shaft and the adapter in the direction of the subject; and wherein the adapter is mounted above a surface of a table by a support member, and the support member is slidably coupled to the table.
